(19) United States
(12) Patent Application Publication
(10) Pub. No.: US 2009/0093810 A1
(43) Pub. Date: Apr. 9, 2009

(54) Electrophysiology Electrodes and Apparatus Including the Same

(76) Inventors: Raj Subramaniam, Fremont, CA (US); Mark D. Mirigian, Hayward, CA (US); Josef V. Koblish, Sunnyvale, CA (US); Leslie A. Oley, Palo Alto, CA (US)

Correspondence Address: HENRICKS SLAVIN AND HOLMES LLP;SUITE 200, 840 APOLLO STREET, EL SEGUNDO, CA, 90245, US

(21) Appl. No.: 12/245,728

(22) Filed: Oct. 4, 2008

Related U.S. Application Data
(60) Provisional application No. 60/978,511, filed on Oct. 9, 2007.

(51) Int. Cl. A61B 18/14

(52) U.S. Cl. 606/41

(57) ABSTRACT

Electrophysiological electrodes including an at least substantially planar distal end and/or surface discontinuities at or adjacent to the distal end are disclosed.

### CROSS-REFERENCE TO RELATED APPLICATIONS

This application claims the benefit of U.S. Provisional Application Ser. No. 60/978,511, filed Oct. 9, 2007 and entitled “Cooled Ablation Catheter Devices and Methods of Use,” which is incorporated herein by reference.

### BACKGROUND

1. Field of the Inventions

The present inventions relate generally to electrodes that may, for example, be used to form lesions in tissue and apparatus including such electrodes.

2. Description of the Related Art

There are many instances where electrodes are inserted into the body. One instance involves the treatment of cardiac conditions such as atrial fibrillation, atrial flutter and ventricular tachycardia, which lead to an unpleasant, irregular heart beat, called arrhythmia. Atrial fibrillation, flutter and ventricular tachycardia occur when anatomical obstacles in the heart disrupt the normally uniform propagation of electrical impulses in the atria. These anatomical obstacles (called “conduction blocks”) can cause the electrical impulse to degenerate into several circular wavelets that circulate about the obstacles. These wavelets, called “reentry circuits,” disrupt the normally uniform activation of the chambers within the heart.

A variety of minimally invasive electrophysiological procedures employing catheters and other apparatus have been developed to treat conditions within the body by ablating soft tissue (i.e. tissue other than blood, bone and connective tissue). With respect to the heart, minimally invasive electrophysiological procedures have been developed to treat atrial fibrillation, atrial flutter and ventricular tachycardia by forming therapeutic lesions in heart tissue. The formation of lesions by the coagulation of soft tissue (also referred to as “ablation”) during minimally invasive surgical procedures can provide the same therapeutic benefits provided by certain invasive, open heart surgical procedures. Atrial fibrillation has, for example, been treated by the formation of one or more long, thin lesions in heart tissue. The treatment of atrial flutter and ventricular tachycardia, on the other hand, requires the formation of relatively large lesions in heart tissue.

The present inventors have determined that conventional methods and apparatus for forming lesions, especially relatively large lesions, are susceptible to improvement. For example, the present inventors have determined that the creation of large lesions with conventional apparatus involves the risk of tissue charring and coagulum formation.

### SUMMARY

An electrode in accordance with one embodiment of a present invention includes a tubular side wall and an at least substantially planar distal wall. An electrode in accordance with another embodiment of a present invention includes a tubular side wall, and end wall, and a plurality of surface discontinuities adjacent to the distal end of the tubular side wall.

Such electrodes provide a number of advantages over conventional electrodes. For example, in those instances where an electrode also includes fluid apertures in the tubular side wall, the planar distal wall and/or the surface discontinuities will create regions of high current density in tissue that is being cooled by the fluid flowing through the apertures.

The above described and many other features and attendant advantages of the present inventions will become apparent as the inventions become better understood by reference to the following detailed description when considered in conjunction with the accompanying drawings.

### BRIEF DESCRIPTION OF THE DRAWINGS

Detailed description of exemplary embodiments will be made with reference to the accompanying drawings.

FIG. 1 is a plan view of a catheter apparatus in accordance with one embodiment of a present invention.

FIG. 2 is a section view take along line 2-2 in FIG. 1.

FIG. 3 is a section view take along line 3-3 in FIG. 1.

FIG. 4 is an elevation view of an electrophysiology electrode in accordance with one embodiment of a present invention.

FIG. 5 is a perspective view of the electrophysiology electrode illustrated in FIG. 4.

FIG. 6 is a section view take along line 6-6 in FIG. 1.

FIG. 7 is an enlarged view of part of the distal portion of the electrophysiology electrode illustrated in FIG. 4.

FIG. 8 is an end view of the electrophysiology electrode illustrated in FIG. 4.

FIG. 9 is a partial section view showing a lesion being formed by the electrophysiology electrode illustrated in FIG. 4.

FIG. 10 is an end view of an electrophysiology electrode with a hemispherical distal end.

FIG. 11 is a partial section view showing a lesion formed by the electrode illustrated in FIG. 10.

FIG. 12 is a partial elevation view of an electrophysiology electrode in accordance with one embodiment of a present invention.

FIG. 13 is an elevation view of an electrophysiology electrode in accordance with one embodiment of a present invention.

FIG. 14 is and end view of the electrophysiology electrode illustrated in FIG. 13.

### DETAILED DESCRIPTION OF EXEMPLARY EMBODIMENTS

The following is a detailed description of the best presently known modes of carrying out the inventions. This description is not to be taken in a limiting sense, but is made merely for the purpose of illustrating the general principles of the inventions.

The present inventions may be used within body lumens, chambers or cavities for diagnostic or therapeutic purposes in those instances where access to interior bodily regions is obtained through, for example, the vascular system or alimentary canal and/or with minimally invasive surgical procedures. For example, the inventions herein have application in the diagnosis and treatment of arrhythmia conditions within the heart. The inventions herein also have application in the diagnosis or treatment of ailments of the gastrointestinal tract, prostrate, brain, gall bladder, uterus, and other regions of the body. With regard to the treatment of conditions within the heart, the present inventions can be used to create lesions to treat atrial fibrillation, atrial flutter and ventricular tachycardia.

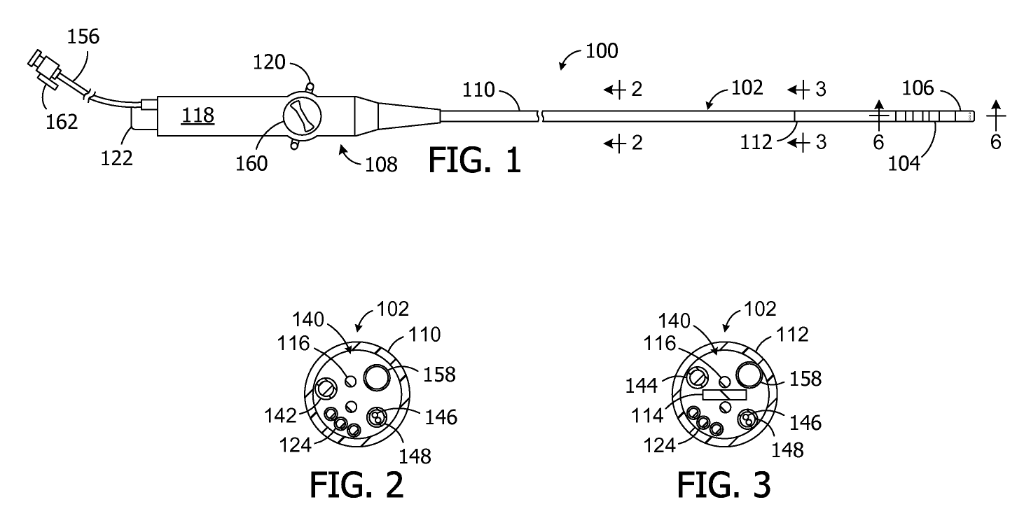

As illustrated for example in FIG. 1, a catheter apparatus 100 in accordance with one embodiment of a present invention includes a hollow, flexible catheter 102, a plurality of ring electrodes 104, a tip electrode 106, and a handle 108. The catheter 102 may be steerable and formed from two tubular parts, or members, both of which are electrically non-conductive. The proximal member 110 is relatively long and is attached to the handle 108, while the distal member 112, which is relatively short, carries the electrodes 104 and 106. The proximal member 110 may be formed from a biocompatible thermoplastic material, such as a Pebax® material (polyether block amide) and stainless steel braid composite or a polyethylene and stainless steel braid composite, which has good torque transmission properties. An elongate guide coil (not shown) may be provided within the proximal member 110. The distal member 112 may be formed from a softer, more flexible biocompatible thermoplastic material such as unbraided Pebax® material, polyethylene, or polyurethane. The proximal and distal members 110 and 112 may be either bonded together with an overlapping thermal bond or adhesively bonded together end to end over a sleeve in what is referred to as a “butt bond.”

Although the present inventions are not so limited, the exemplary catheter 102 is configured for use within the heart and, accordingly, is about 6 French to about 10 French in diameter. The portion of the catheter 102 that is inserted into the patient is typically from about 60 to 160 cm in length. The length and flexibility of the catheter 102 allow the catheter to be inserted into a main vein or artery (typically the femoral vein), directed into the interior of the heart, and then manipulated such that the desired electrode(s) 104 and/or 106 contact the target tissue. Fluoroscopic imaging may be used to provide the physician with a visual indication of the location of the catheter 102.

With respect to steering, the exemplary catheter apparatus 100 illustrated in FIGS. 1-3 may be provided with a conventional steering center support and steering wire arrangement. The proximal end of the exemplary steering center support 114 is mounted near the distal end of the proximal member 110, while the distal end of the steering center support is secured to (but electrically insulated from) the tip electrode 106 in the manner described below. A pair of steering wires 116 are secured to opposite sides of the steering center support 114 and extend through the catheter body 102 to the handle 108, which is also configured for steering. More specifically, the exemplary handle 108 includes a handle body 118 and a lever 120 that is rotatable relative to the handle body. The proximal end of the catheter 102 is secured to the handle body 118, while the proximal ends of the steering wires 116 are secured to the lever 120. Rotation of the lever 120 will cause the catheter distal member 112 to deflect relative to the proximal member 110. Additional details concerning this type of steering arrangement may be found in, for example, U.S. Pat. Nos. 5,871,525 and 6,287,301. Other suitable steering arrangements are disclosed in U.S. Pat. Nos. 6,013,052 and 6,287,301. Nevertheless, it should be noted that the present inventions are not limited to steerable catheter apparatus, or to any particular type of steering arrangement in those catheter apparatus which are steerable.

The exemplary ring electrodes 104, which may be used for electrical sensing or tissue ablation, are connected to an electrical connector 122 on the handle 108 by signal wires 124. Electrically conducting materials, such as silver, platinum, gold, stainless steel, plated brass, platinum iridium and combinations thereof, may be used to form the electrodes 104. The diameter of the exemplary electrodes 104 will typically range from about 5 French to about 11 French, while the length is typically about 1 mm to about 4 mm with a spacing of about 1 mm to about 10 mm between adjacent electrodes. The ring electrodes 104 may also, for example, be replaced by conductive coils, replaced by some other tissue heating device, or simply omitted. Temperature sensors (not shown) may also be associated with the ring electrodes 104 and connected to the electrical connector 122 by signal wires.

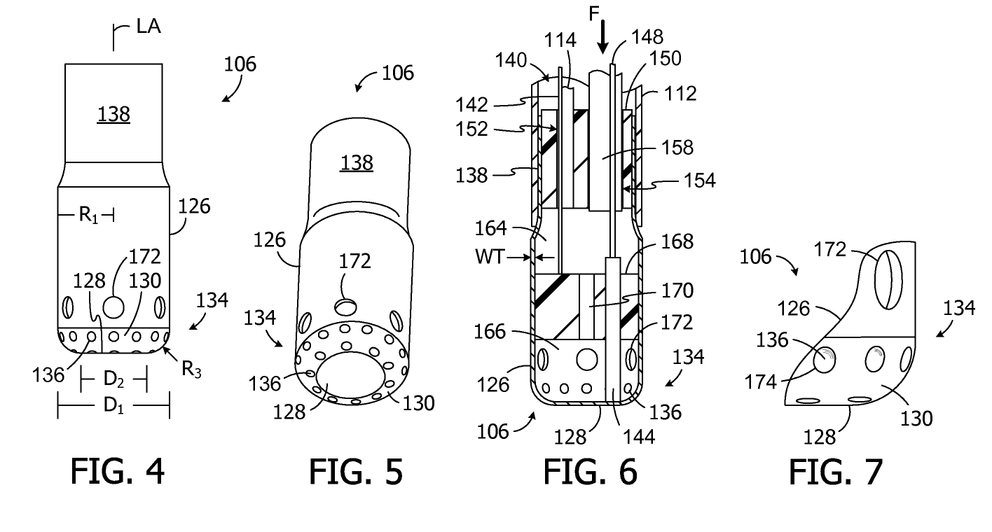

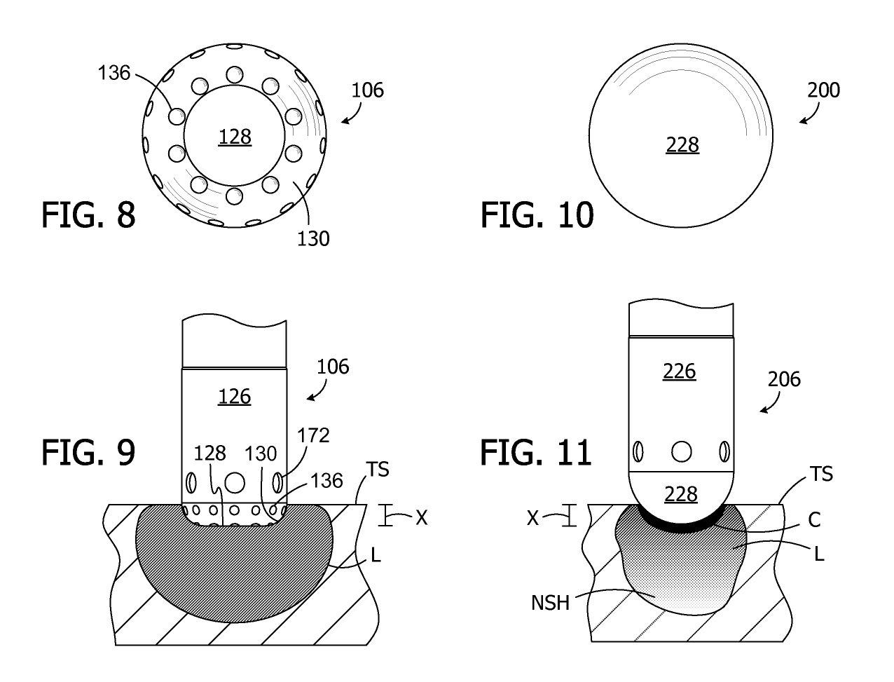

Turning to FIGS. 4-7, the exemplary tip electrode 106 includes a tubular side wall 126, a planar end wall 128 and a curved wall 130 that extends from the side wall to the end wall. The distal region 134 of the tip electrode 106 may have a plurality of surface discontinuities 136. In the illustrated embodiment, the surface discontinuities 136 are generally hemispherical in shape and are located on the curved wall 130. The respective advantages associated with the shape of the planar end wall 128 and the surface discontinuities 136, e.g. spreading current over a relatively large tissue contact area and concentrating current in advantageous locations, are discussed below with reference to FIGS. 8-11.

The exemplary tip electrode 106 illustrated in FIGS. 4-7 is configured to be inserted into the distal end of the catheter distal portion 112 (or other electrophysiology apparatus) and secured thereto catheter with adhesive or some other suitable instrumentality or method. To that end, the tubular side wall 126 includes a proximal region 138 of reduced width that is configured to fit into the catheter lumen 140. By way of example, but not limitation, the tip electrode may, in other implementations, be configured for a butt end connection or configured to extend over the distal end of the catheter. Power for the tip electrode 106 is provided by a power wire 142 (FIGS. 2, 3 and 6) that is soldered to a portion of the tip electrode and extends through the catheter lumen 140 to the electrical connector 122 on the handle 108. A temperature sensor 144 may be mounted within the electrode 106 and, in the illustrated embodiment, the temperature sensor is a thermocouple. The thermocouple wires 146 extend through a tube 148 (FIGS. 2, 3 and 6) to the electrical connector 122.

As illustrated in FIG. 6, an anchor member 150 may be mounted within the proximal region 138 of the exemplary electrode 106. The anchor member 150, which may be formed from an electrically conductive material such as stainless steel or an electrically non-conductive material such as nylon or polyimide, includes a pair of lumens 152 and 154. The steering center support 114 is positioned within the lumen 152 and is secured to the anchor member 150. In those instances where the anchor member 150 is electrically conductive, the portion of the steering center support 114 secured thereto may be covered with an electrically non-conductive material. The power wire 142 extends through the lumen 152, while the thermocouple tube 148 extends through the lumen 154. Additionally, in those instances where a steering center support is not employed, a single steering wire may be secured to the anchor member 150.

The exemplary catheter apparatus 100 is also capable of employing fluid to cool the tip electrode 106 and to cool tissue that is adjacent to certain portions of the tip electrode. Referring first to FIGS. 1-3, a fluid inlet tube 156 extends into the handle 108 and is connected to a valve (not shown) within the handle. A fluid tube 158 extends from the valve to the tip electrode 106. A control knob 160 on the handle body 118 is connected to the valve and allows the clinician to control the fluid flow rate through the valve. A connector 162, which may be connected to a source of cooling fluid, is mounted on the proximal end of the fluid tube 156. Turning to FIG. 6, the distal end of the fluid tube 158 is mounted within the anchor member lumen 154 in the illustrated embodiment.

The tip electrode 106 may be configured such that there are one or more cooling chambers into which cooling fluid is delivered. In the illustrated embodiment, and referring to FIG. 6, the tip electrode 106 includes a pair of cooling chambers 164 and 166 that are separated by a thermal mass 168. Cooling fluid F enters the cooling chamber 164 by way of the fluid tube 158. A fluid lumen 170 in the thermal mass 168 allows fluid to flow from the cooling chamber 164 to the cooling chamber 166. Fluid exits the cooling chamber 166 (and the electrode 106) by way of a plurality of fluid outlets 172 that are aligned with the cooling chamber 166 and extend through the tubular side wall 126. The fluid outlets 172 may be located immediately proximal to the distal region 134 and, in the illustrated embodiments, are about 1 mm to 3 mm from the distal end of the tip electrode 106.

The cooling fluid cools both the tip electrode 106 and the tissue adjacent to the perimeter of the tip electrode. For example, the cooling fluid draws heat from the tip electrode 106 (including the thermal mass 168) and reduces the temperature of the electrode. The presence of the cooling chambers 164 and 166 augments the fluid cooling because the fluid circulates within the cooling chamber 164 prior to entering the cooling chamber 166, and circulates within the cooling chamber 166 prior to exiting the tip electrode 106 by way of the fluid outlets 172. The decrease in electrode and tissue temperature reduces the likelihood that the tissue in contact with the tip electrode 106 will char and/or that coagulum will form on the surface of the tip electrode. As such, the amount of energy supplied to the tissue may be increased, and the energy is transferred to the tissue more efficiently, as compared to an electrode that is not configured for fluid cooling. This results in the formation of larger and deeper lesions. In addition to cooling tissue adjacent to the tip electrode 106, fluid that exits the tip electrode sweeps biological material such as blood and tissue away from the electrode, further reducing the likelihood of coagulum formation.

As alluded to above, there are a variety of advantages associated with the planar end wall 128 and surface discontinuities 136. At least some of the advantages may be explained by comparing the exemplary tip electrode to an otherwise identical electrode with a hemispherical end wall and no surface discontinuities (hereafter “hemispherical electrode”). Accordingly, FIGS. 8 and 10 are end views of the exemplary tip electrode 106 and a hemispherical electrode 206 with a hemispherical end wall 228, and FIGS. 9 and 11 are partial section views showing lesions being formed with the exemplary tip electrode 106 and the hemispherical electrode 206.

The surface area of the exemplary tip electrode 106 that is in contact with tissue is larger than the surface area of the hemispherical electrode 206 that is in contact with tissue when both electrodes are pushed the same distance X into the tissue surface TS. As such, the current density associated with the exemplary tip electrode 106 is less than that of the hemispherical electrode 206 and, accordingly, the exemplary tip electrode 106 is less likely than the hemispherical electrode 206 to cause tissue charring and coagulum formation. There is also a more abrupt transition between the side wall 126 and the portion of the tip electrode 106 that is in contract with tissue than there is in the hemispherical electrode 206. In the illustrated embodiment, the abrupt transition is provided by the relatively small radius of curvature of the curved wall 130 and the intersection of the curved wall and the planar end wall 128. The abrupt transition associated with the exemplary tip electrode 106 is also located near the outer perimeter of the electrode, i.e. the outer perimeter of the tubular wall 126 taken in plane perpendicular to the longitudinal axis LA (FIG. 4). The “edge effect” associated with the abrupt transition draws more current to outer perimeter of the electrode 106, which results in more current being delivered to the tissue that is being cooled by the fluid flowing from the fluid outlets 172 than to the tissue that is closer to the center of the planar end wall 128. Directing more of the current to the tissue that is being cooled further reduces the likelihood, as compared to the hemispherical electrode 206, that an ablation procedure will result in tissue charring and coagulum formation.

The surface discontinuities 136, each of which includes an edge 174 (FIG. 7), are located adjacent to the planar end wall 128 so that they will be in contact with tissue that is adjacent to the fluid outlets 172 when the electrode is pressed into tissue surface TS. To that end, the surface discontinuities 136 are located on the curved wall 130 in the illustrated embodiment. The edges 174 each create an “edge effect” that draws more current than would be the case if the edges were not present. The locations of the “edge effects” created by the discontinuities results in more current being delivered to the tissue at the outer perimeter of the electrode 106, which is being cooled by the fluid flowing from the fluid outlets 172, than to the tissue that is closer to the center of the planar end wall 128. Here too, directing more of the current to the tissue that is being cooled reduces the likelihood, as compared to an electrode such as the hemispherical electrode 206 without discontinuities, that an ablation procedure will result in tissue charring and coagulum formation.

In the comparison presented in FIGS. 9 and 11, the exemplary tip electrode 106 and the hemispherical electrode 206 are pressed into the tissue surface TS the same distance X, the same amount of current being supplied to the electrodes, and cooling fluid is being supplied at the same rate. The magnitude of the current is slightly below that which would result in char of the tissue being ablated by the tip electrode 106 and/or the formation of substantial coagulum thereon. The lesion L produced by the exemplary tip electrode 106 is wider and deeper than the lesion L produced by the hemispherical electrode 206. With respect to depth, note that the exemplary tip electrode 106 also created a more uniform lesion (as emphasized by the uniform coloring in FIG. 9) and that the tissue associated with the hemispherical electrode 206 includes a region NSH that is not sufficiently heated, due to the formation of char C, to create a lesion.

It should also be noted here that the exemplary tip electrode 106 need not be perpendicular (FIG. 9) to the tissue surface to realize the beneficial effects described above. At any orientation relative to the tissue surface, i.e. from perpendicular to parallel, the “edge effect” associated with abrupt transition from the tubular wall 126 to the planar end wall 128, and/or the “edge effects” associated with the surface discontinuities 136, will result in more current being delivered to the tissue that is being cooled by the fluid flowing from the fluid outlets 172. As noted above, directing more of the current to the tissue that is being cooled reduces the likelihood that an ablation procedure will result in tissue charring and coagulum formation.

With respect to material, the exemplary tip electrode 106 may be formed from any suitable electrically conductive material. By way of example, but not limitation, suitable materials for the main portion of the tip electrode 106, i.e. the tubular side wall 126, a planar end wall 128 and a curved wall 130, include silver, platinum, gold, stainless steel, plated brass, platinum iridium and combinations thereof. The thermal mass 168 may be formed from any suitable electrically and thermally conducting material such as, for example, brass, copper and stainless. The thermal mass 168 may, alternatively, be made of thermally conducting and electrically non-conducing materials. Here, the power wire 142 will be attached to another portion of the tip electrode 106, e.g. tubular side wall 126.

Turning to shape and dimension, the exemplary tip electrode 106 is generally cylindrical in shape and is sized for use within the heart. To that end, the outer diameter D1 (FIG. 4) of the tubular side wall 126 may be from about 5 French to about 11 French (about 1.67 mm to about 3.67 mm) and the length of the tubular side wall may be about 2 mm to about 6 mm, with about 30% occupied by the proximal region 138. It should be noted, however, that the present tip electrodes are not limited to a circular cross-section. The wall thickness WT (FIG. 6) of the exemplary tip electrode 106 may be about 0.05 mm to about 0.3 mm.

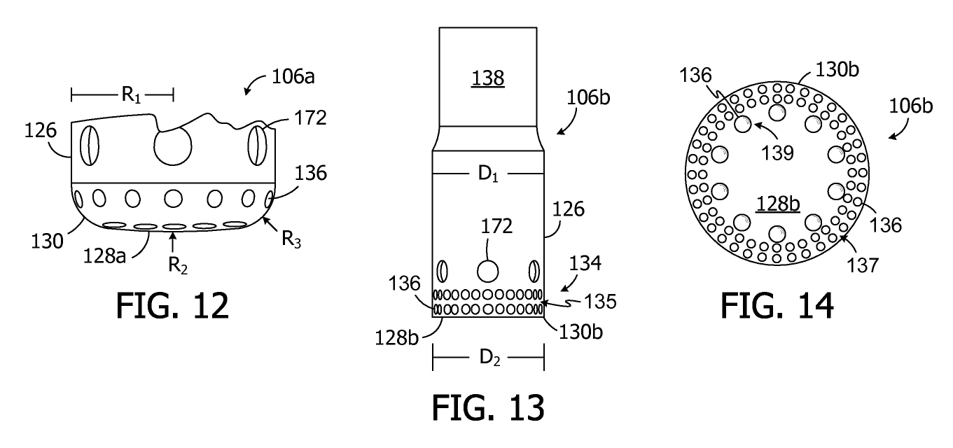

The diameter D2 of the planar end wall 128 may be about 30% to about 95% of the diameter of the outer diameter D1 of the tubular side wall 126 when the curved wall 130 (or other transitional wall or surface) is present and in some implementations may be about 60% to about 90%. The end wall 128 may be planar as shown in FIGS. 4-9, i.e. flat, or may be at least substantially planar. Referring to FIG. 12, and as used herein, an “at least substantially planar” end wall (e.g. end wall 128a on the electrode 106a that is otherwise identical to electrode 106) is an end wall with a radius of curvature R2 that is at least 3 times the radius R1 of tubular side wall from which it extends, and may range from about 3 times the radius R1 to about 6 times the radius R1 in some implementations. For purposes of comparison, the radius of curvature of a hemispherical end wall such as hemispherical end wall 228 (FIGS. 10 and 11) is equal to the radius of the tubular side wall from which it extends, while the radius of curvature of a flat wall is infinite. The radius R3 of the curved wall 130 (FIG. 4), which defines a 90 degree arc in the illustrated embodiment, may be about 20% to about 60% of the radius R1 of the tubular sidewall 126.

The curved wall 130 may be also eliminated from embodiments including, but not limited to, those that are otherwise identical to the embodiments described above with reference to FIGS. 4-9 and 12. In the exemplary tip electrode 106b illustrated in FIGS. 13 and 14, which is otherwise identical to tip electrode 106, the curved wall 130 has been replaced by a corner 130b at the intersection of the tubular side wall 126 and a planar end wall 128b. Here, the outer diameter D2 of the planar end wall 128b will be equal to the diameter of the outer diameter D1 of the tubular side wall 126. Surface discontinuities 136 may be located on the tubular side wall 126, the planar end wall 128b, or both (as shown). Still another alternative is to replace the curved wall 130 and/or corner 130b with a chamfer-like wall or other transition (not shown) that extends from the tubular side wall to the planar end wall. Surface discontinuities may be provided on such a transition.

The axial length of distal region 134 of the tip electrode 106, i.e. the region that is distal of the fluid outlets, may be about 0.2 mm to about 1 mm. The distal region 134 may include some or all of the curved wall 130, chamfer or other transition, if present, or a portion of the tubular side wall 126 in those instances where a corner 130b is present. It should also be noted that, in those implementations where it is intended that the fluid outlets 172 be close to the tissue surface during lesion formation procedures, the distal ends of the fluid outlets will be about 0.5 mm to about 2 mm from the end wall 128-128b.

Turning to the surface discontinuities, and although the present inventions are not limited to any particular shape or size, the surface discontinuities 136 in the illustrated embodiments are hemispherical-shaped indentations in the tip electrode wall that are about 0.1 mm to about 0.5 mm in depth and diameter. Depending on size and the method of manufacture, the surface discontinuities 136 may result in corresponding discontinuities on the inner surface of the electrode (FIG. 6). The surface discontinuities 136 may be positioned on the distal region 134 so that they will be in contact with tissue, and may cover about 30% to about 70% of the associated portion of the electrode surface, depending on the intended effect. The surface discontinuities 136 within any particular tip electrode, or portion thereof, may be of uniform size and density or may vary in size and/or density. Referring for example to FIGS. 7-9, the surface discontinuities 136 on the exemplary tip electrode 106 may be arranged in two groups within the curved wall 130 and the distal region 134. One group is just distal of the tubular side wall 126 and the other group is just proximal of the planar end wall 128. All of the surface discontinuities 136 are the same size and the density of each group is essentially the same. Turning to exemplary tip electrode illustrated in FIGS. 13 and 14, there is a relatively high density group 135 of relatively small surface discontinuities 136 on the tubular side wall 126 within the distal region 134, and a relatively high density group 137 of relatively small surface discontinuities near the outer perimeter of the planar end wall 128b. The relative high density discontinuity groups 135 and 137 will produce higher current density near the outer perimeter of the distal region 134 than would, for example, the lower density groups illustrated in FIGS. 7-9. There is also a relatively low density group 139 of relatively large surface discontinuities 136 radially inward of the high density group 137 on the planar end wall 128b. Although the current density at this portion of the tip electrode 106b will be greater than it would near the center of the planar end wall 128b, the current density will be lower than radially outward portion occupied by the higher density group 137.

Surface discontinuities are also not limited indentations. By way of example, but not limitation, the distal region 134 of tip electrodes in accordance with some embodiments may be provided with surface protrusions, such as hemispherical surface protrusions.

It should also be noted that there are no holes in the end walls of the exemplary tip electrodes 106-106b for fluid cooling and/or passage of a temperature sensor that is aligned with the outer surface of the electrode. Such holes would, like the surface discontinuities 136, creates regions of high current density and regions of high current density near the center of the tip electrodes would work against the above-described efforts to move current to the outer perimeter of the tip electrodes.

Although the present inventions have been described in terms of the preferred embodiments above, numerous modifications and/or additions to the above-described preferred embodiments would be readily apparent to one skilled in the art. By way of example, but not limitation, catheter apparatus may be configured such that some of the cooling fluid is returned to the fluid source by way of a second fluid tube. The present inventions are also applicable to surgical probes with relatively short shafts. It is intended that the scope of the present inventions extend to all such modifications and/or additions and that the scope of the present inventions is limited solely by the claims set forth below.

1. An electrophysiology electrode, comprising:
a tubular side wall defining a distal end and including at least one fluid aperture; and
an at least substantially planar distal wall, without an aperture extending therethrough, associated with the distal end of the tubular side wall.

2. An electrophysiology electrode as claimed in claim 1, wherein the at least one fluid aperture comprises a plurality of fluid apertures.

3. An electrophysiology electrode as claimed in claim 1, wherein the tubular side wall defines an annular cross-section.

4. An electrophysiology electrode as claimed in claim 1, wherein the tubular side wall and the at least substantially planar distal wall together define a distal corner.

5. An electrophysiology electrode as claimed in claim 4, further comprising:
a plurality of surface discontinuities adjacent to the distal corner.

6. An electrophysiology electrode as claimed in claim 5, wherein the plurality of surface discontinuities are associated with the side wall and/or the at least substantially planar distal wall.

7. An electrophysiology electrode as claimed in claim 5, wherein the plurality of surface discontinuities comprises a plurality of partially spherical indentations.

8. An electrophysiology electrode as claimed in claim 1, further comprising:
a curved surface located between the tubular side wall and the at least substantially planar distal wall.

9. An electrophysiology electrode as claimed in claim 8, further comprising:
a plurality of surface discontinuities associated with the curved surface.

10. An electrophysiology electrode as claimed in claim 9, wherein the plurality of surface discontinuities comprises a plurality of partially spherical indentations.

11. An electrophysiology electrode as claimed in claim 1, wherein the at least substantially planar distal wall comprises a flat distal wall.

12. An electrophysiology electrode, comprising:
a tubular side wall defining a distal end;
a distal wall associated with the distal end of the tubular side wall; and
a plurality of surface discontinuities adjacent to the distal end of the tubular side wall.

13. An electrophysiology electrode as claimed in claim 12, wherein the plurality of surface discontinuities comprises a plurality of partially spherical indentations.

14. An electrophysiology electrode as claimed in claim 12, wherein the plurality of surface discontinuities are on the tubular side wall.

15. An electrophysiology electrode as claimed in claim 12, wherein the plurality of surface discontinuities are on the end wall.

16. An electrophysiology electrode as claimed in claim 12, wherein the plurality of surface discontinuities are on the tubular side wall and the end wall.

17. An electrophysiology electrode as claimed in claim 12, further comprising:
a curved surface located between the tubular side wall and the distal wall;
wherein the plurality of surface discontinuities are on the curved surface.

18. An electrophysiology electrode as claimed in claim 12, wherein
the distal wall defines the distal end of the electrophysiology electrode; and
the surface discontinuities are located no more than 1 mm from the distal end of the electrophysiology electrode.

19. An electrophysiology electrode as claimed in claim 12, wherein the surface discontinuities are arranged in first group having a first density and a second group having a second density greater than the first density.

20. An electrophysiology electrode as claimed in claim 19, wherein the second group is located between the first group and the longitudinal end of the tubular side wall.

21. An electrophysiology electrode as claimed in claim 12, further comprising:
a plurality of fluid apertures in the tubular side wall.

22. An electrophysiology electrode for use with soft tissue, the electrophysiology electrode comprising:
a tubular side wall defining a distal end;
a distal wall, associated with the distal end of the tubular side wall, defining a central region and an outer perimeter that extends around the central region; and
means, associated with the tubular side wall and/or the distal wall, for creating higher current density in soft tissue adjacent to the outer perimeter of the distal wall than in soft tissue adjacent to the central region of the distal wall when current flows through the electrophysiology electrode to soft tissue in contact with the electrophysiology electrode.

23. An electrophysiology electrode as claimed in claim 22, further comprising:
a plurality of fluid apertures in the tubular side wall.

24. (canceled)
